(19) United States
(12) Patent Application Publication
(10) Pub. No.: US 2026/0355768 A1
(43) Pub. Date: Nov. 12, 2026

(54) PUMP

(71) Applicant: APTAR FRANCE SAS, Le Neubourg (FR)

(72) Inventors: David FABIEN, Saint Renan (FR); Fabrizio CAMPLONE, Pescara (PE) (IT); Marco ZAVARELLA, Sulmona (Aq) (IT)

(73) Assignee: APTAR FRANCE SAS, Le Neubourg (FR)

(21) Appl. No.: 19/103,064

(22) PCT Filed: Aug. 9, 2023
(86) PCT No.: PCT/FR2023/051259

§ 371 Date: Feb. 11, 2025

(30) Foreign Application Priority Data
Aug. 12, 2022 (FR) FR2208276

(51) Int. Cl. B05B 11/10

(52) U.S. Cl. CPC B05B 11/1078; B05B 11/1023; B05B 11/1059

(57) ABSTRACT

A pump having a fluid product chamber, an actuating pin and a return mechanism for returning the actuating pin to an inoperative position, the actuating pin can be actuated by a user. The return mechanism has a partial vacuum chamber having a volume variable between a minimum volume state and a maximum volume state by displacing the actuating pin, the minimum volume state obtained when the actuating pin is in the inoperative position and the maximum volume state obtained when the actuating pin is in the depressed position. The maximum volume state corresponds to a maximum partial vacuum and the minimum volume state corresponds to a minimum partial vacuum, the actuating pin being urged towards its inoperative position by the partial vacuum prevailing in the partial vacuum chamber.

The present invention relates to a pump comprising a fluid product chamber with a variable volume, as well as an actuating pin which is axially displaceable between an inoperative position and a depressed position, in a manner such as to vary the volume of the chamber respectively between a state of maximum volume and a state of minimum volume. The pump comprises a return means for returning the actuating pin to the inoperative position, corresponding to the maximum volume state of the fluid product chamber. The actuating pin is provided with an actuator which can be actuated by a user. Advantageous fields of application of the invention are those of pharmacy, cosmetics and perfumery, without excluding others.

Conventionally, the return means is in the form of a spring which is generally produced from metal. The problem with these metal springs is that they impede or complicate recycling of the pump, all the more so because they are disposed in the pump body. Plastic springs which are easier to recycle exist, but they tend to lose their stiffness over time.

The present invention seeks to overcome the disadvantages of these prior art springs by proposing another type of elastic return means which presents no problems as regards recycling or stiffness.

To this end, the present invention proposes that the return means comprises a partial vacuum chamber, the volume of which varies between a state of minimum volume and a state of maximum volume by displacement of the actuating pin, the state of minimum volume of the partial vacuum ¹ Translation of the title as established ex officio. chamber being reached in the inoperative position of the actuating pin and the state of maximum volume of the partial vacuum chamber being reached in the depressed position of the actuating pin, the state of maximum volume of the partial vacuum chamber corresponding to a maximum partial vacuum and the state of minimum volume of the partial vacuum chamber corresponding to a minimum partial vacuum, the actuating pin being urged towards its inoperative position by the partial vacuum prevailing in the partial vacuum chamber.

The partial vacuum chamber thus generates a partial vacuum from a point at which the pressure of the partial vacuum chamber is close to or equal to atmospheric pressure. The pressure in the partial vacuum chamber is therefore always less than or equal to atmospheric pressure. The partial vacuum increases as the actuating pin is displaced from its inoperative position towards its depressed position. It is a type of reverse air pump.

In accordance with the invention, the pump further comprises mechanical means, which are independent of the partial vacuum in the partial vacuum chamber, which can be actuated by the user in order to return and maintain the partial vacuum chamber in its state of minimum volume. Thus, starting from the state of maximum volume (maximum partial vacuum), the partial vacuum chamber will decrease in volume as soon as the user releases the pressure on the actuator. This decrease in volume under the action of the partial vacuum continues until the mechanical means are engaged. Consequently, the user can actuate these mechanical means in order to further reduce the volume of the partial vacuum chamber to finally reach its minimum volume state. The partial vacuum in the state of minimum volume does not need to be present during the entirety of the sequence for actuating the mechanical means. It suffices for the partial vacuum to trigger the mechanical means.

Advantageously, the mechanical means are controlled by the actuator via the actuating pin. In other words, the user grasps the actuator and displaces it, which has the effect of actuating the mechanical means. The displacement of the actuator may be rotary, axial, or both at the same time. In accordance with the invention, the mechanical means comprise a threaded connection, which makes it possible to displace the actuator and the actuating pin both axially and in rotation. Advantageously, the threaded connection can define an axial travel, which is delimited on one side by a top abutment corresponding to the minimum volume state of the partial vacuum chamber and on the other side by a release of the threaded connection, allowing subsequent variation of the volume of the partial vacuum chamber towards its maximum volume state. From an ergonomic and gestural point of view, the user will grasp the actuator to rotate it through a specific angle, for example a quarter turn or a half turn, in order to bring it selectively into the locked or unlocked position. This locking/unlocking system by rotating the actuator is conventional. In the context of the invention, the actuation of the locking/unlocking system also simultaneously causes the actuation of the mechanical means, without the user even perceiving it.

Because of the mechanical means, the partial vacuum chamber always returns to the same minimum volume starting point.

In accordance with another advantageous characteristic of the invention, which can advantageously be combined with mechanical means, the partial vacuum chamber communicates with the outside in its state of minimum volume. Thus, even if the partial vacuum chamber leaks and allows air to enter, this air will be evacuated to the outside, so that the partial vacuum chamber always starts at the same pressure, which is close to atmospheric pressure. This initial or final leakage can be implemented independently of the mechanical means, but it is then necessary for the partial vacuum in the partial vacuum chamber to be sufficient to bring the partial vacuum chamber into or close to its state of minimum volume. This is possible, but difficult to achieve in practice and especially in a repeatable manner. This is why this initial or final leakage is of particular advantage in combination with the mechanical means, guaranteeing that the partial vacuum chamber always returns to its state of minimum volume. It can then be said that the mechanical means supplement or complete the partial vacuum in order to ensure that the partial vacuum chamber is reset at each actuation cycle by connecting it to the outside.

In accordance with one embodiment of the invention, the actuating pin comprises a partial vacuum chamber piston which slides axially in a sealed manner in a barrel of the partial vacuum chamber, in a manner such as to define the partial vacuum chamber between them, the actuating pin forming a venting groove which brings the partial vacuum chamber into communication with the outside in the inoperative state of the actuating pin, which corresponds to the minimum volume state of the partial vacuum chamber. The groove, flute or passage establishes air communication between the partial vacuum chamber and the outside when the pump is inactive, this communication being cut off in a manner such as to isolate the partial vacuum chamber as soon as the actuating pin leaves its inoperative state or shortly after leaving its inoperative state. A travel of a few tenths of a millimetre is sufficient to isolate the partial vacuum chamber.

In accordance with another aspect of the invention, the actuating pin may comprise a piston member and a valve shutter, the piston member forming the partial vacuum chamber piston sliding in the barrel of the partial vacuum chamber, the piston member also forming a fluid product chamber piston sliding in a fluid product chamber barrel, the piston member advantageously forming a movable outlet valve member for the fluid product chamber, and the valve shutter advantageously forming an outlet valve seat for the fluid product chamber. Advantageously, the piston member is axially movable with respect to the valve shutter under the effect of the pressure prevailing in the fluid product chamber, but is driven in rotation by the valve shutter. Advantageously, the threaded connection is located between the piston member and the barrel of the partial vacuum chamber. The piston member may be termed a differential piston, in that it is displaced under the effect of the pressure prevailing in the fluid product chamber. Instead of this piston member, it is possible to provide an actuating pin provided with a fixed partial vacuum chamber piston and a fluid product chamber piston which slides on the actuating pin under the effect of the pressure prevailing in the fluid product chamber.

In accordance with another approach of the invention, it could be said that the partial vacuum chamber defines a maximum axial travel between its state of minimum volume and its state of maximum volume, this maximum axial travel being broken down into:
a major travel starting from the maximum volume state, which is induced by the partial vacuum prevailing in the partial vacuum chamber, and
a minor travel to the state of maximum volume, which is induced by driving the actuator in rotation and/or axially, which ends in a position of axial locking of the actuator.

The user, who grasps a dispenser on which the pump of the invention is mounted, will quickly discern that the actuator is in the locked position by pressing the top or by taking note of a visible instruction on the actuator. The user will therefore drive the actuator in rotation through a specific angle in order to unlock it. In doing so, but imperceptibly, the user has actuated the mechanical means, which have brought the partial vacuum chamber from its state of minimum volume, in which it communicates with the outside, to an intermediate state in which it is isolated from the outside. In the case in which the mechanical means employ a threaded connection, the intermediate position corresponds to the end of the threaded connection, in which the actuating pin is released from this threaded connection. The user then presses axially on the actuator, which has the simultaneous effects of dispensing fluid product and generating an increasing partial vacuum in the partial vacuum chamber. Once dispensing of fluid product has been completed, the user releases the axial pressure on the actuator, so that the partial vacuum in the partial vacuum chamber has the effect of returning the actuating pin and its actuator towards the inoperative position. However, the return travel of the rod is stopped in the intermediate position, which corresponds to the activation of the mechanical means. In the case in which the mechanical means employ a threaded connection, the partial vacuum raises the actuating pin until the threads come into engagement. It is then sufficient for the user to turn the actuator to lock it, which simultaneously has the effect of returning the actuating pin to the inoperative position and the partial vacuum chamber to its state of minimum volume in which it communicates with the outside through the venting groove of the actuating pin. The locked position corresponds to an abutment of the threaded connection.

The spirit of the invention resides in the threaded connection which allows the air spring to return to its starting point. In addition, in order to mitigate a risk of leakage, it is vented at its starting point. The threaded connection combined with the leakage at the starting point provides a synergistic effect, making its operation entirely repeatable, and therefore reliable. The air spring is thus reset after each actuation.

The invention will now be described in more detail with reference to the accompanying drawings, given by way of non-limiting example, of an embodiment of the invention. In the figures:

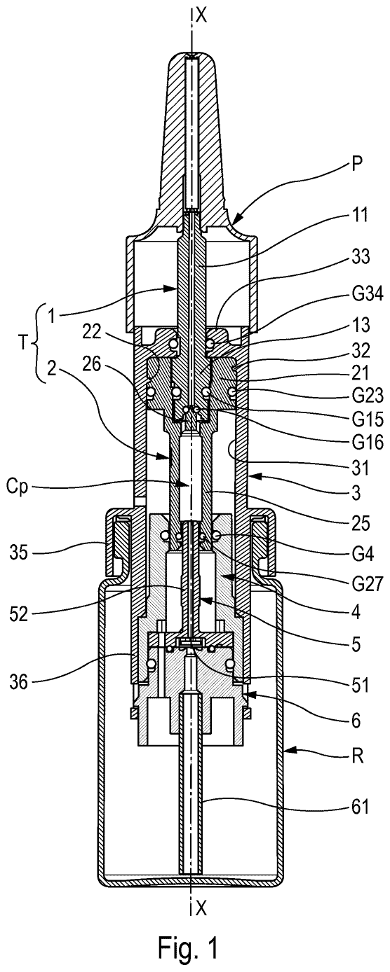

FIG. 1 is a vertical cross-sectional view through a fluid product dispenser in accordance with the invention, locked and inoperative,

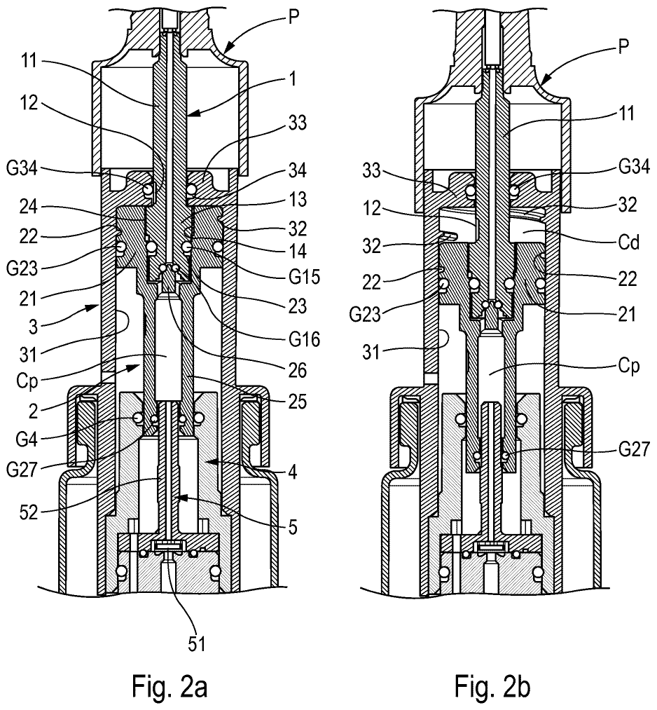

FIG. 2a is an enlarged view of FIG. 1 with the pump, locked and inoperative,

FIG. 2b is a view similar to FIG. 2a with the pump unlocked,

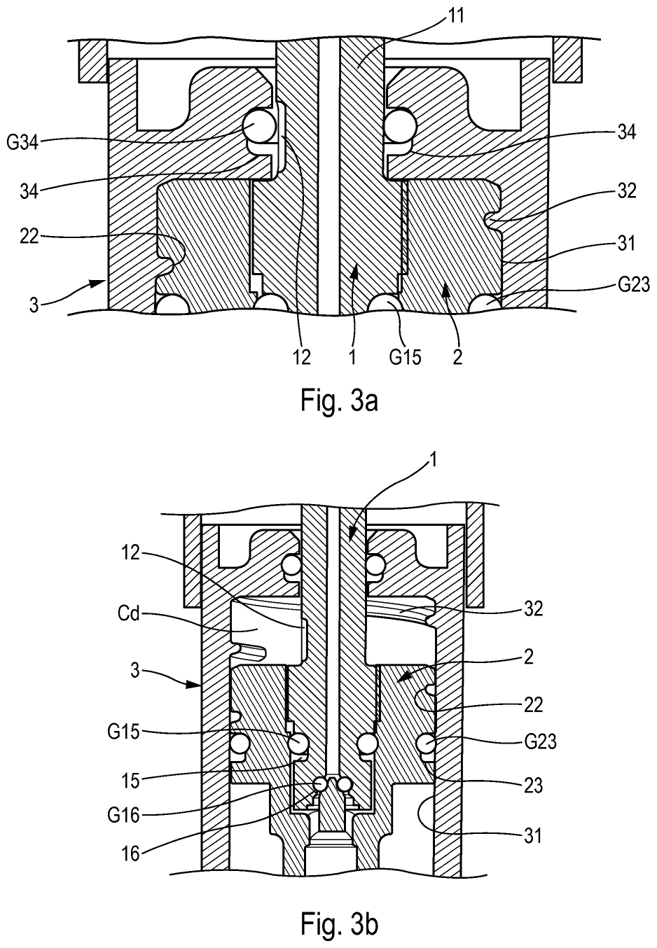

FIG. 3a is a greatly enlarged view of a portion of FIG. 2a,

FIG. 3b is a greatly enlarged view of a portion of FIG. 2b,

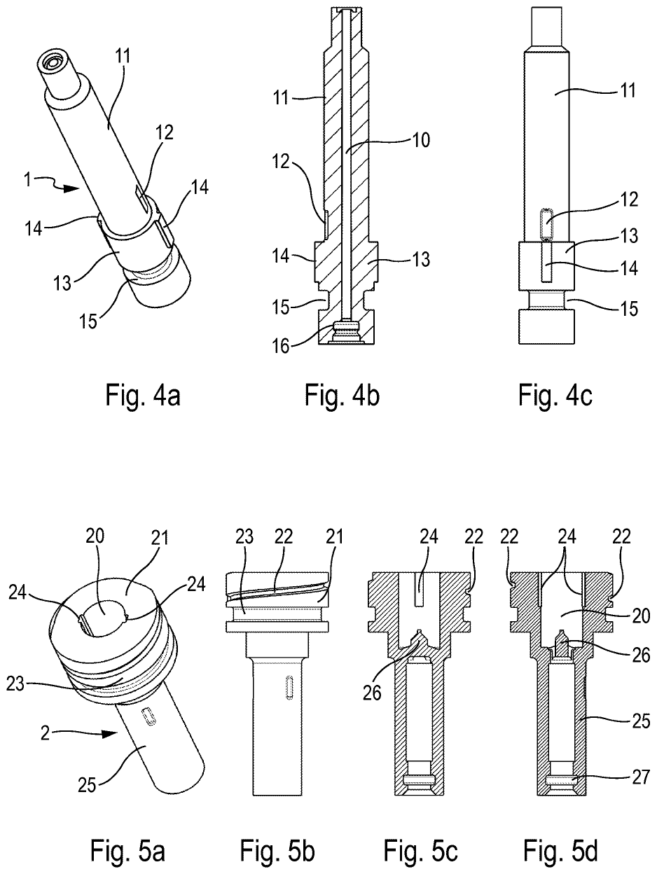

FIGS. 4a, 4b and 4c represent the valve shutter of the pump in accordance with the invention,

FIGS. 5a, 5b, 5c and 5d represent the piston member of the pump in accordance with the invention, and

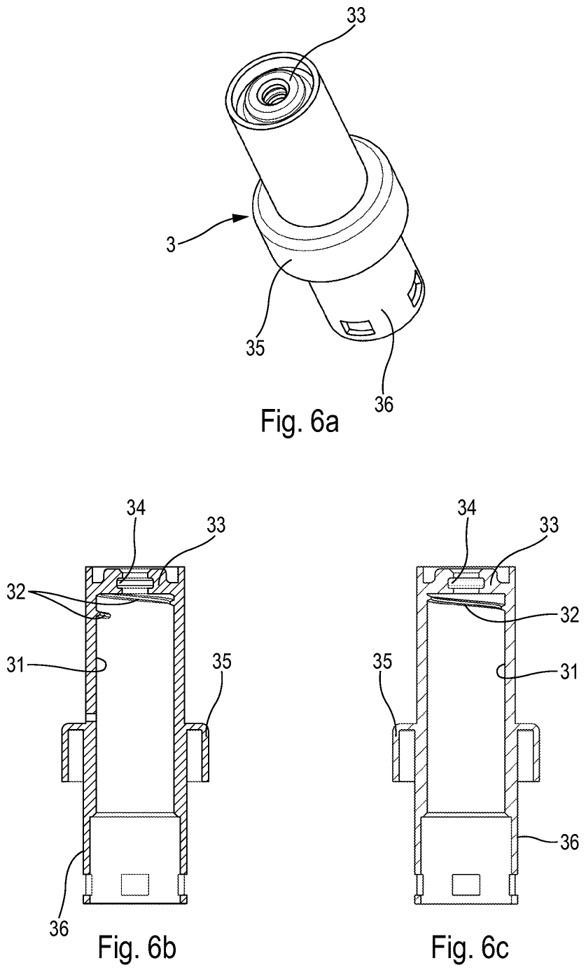

FIGS. 6a, 6b and 6c represent the pump body in accordance with the invention.

FIG. 1 shows the pump of the invention mounted on a fluid product reservoir R, which is not critical for the present invention. In fact, this reservoir R may be of any type, of any shape, and produced from any appropriate material. It is sufficient for it to form an opening, for example in the form of a neck, on which the pump of the invention can be mounted in a secure and sealed manner.

The pump of the invention comprises first of all a pump body 3 which receives fixed elements 4, 5 and 6 as well as a movable element, namely an actuating pin T. The pump body 3 forms a ring 35 intended to cooperate with the neck of the reservoir R for securing the pump to the reservoir. Below this fixing ring 35, the pump body 3 forms an assembly bushing 36, in which a bottom part 6 provided with a dip tube 61, a barrel part 5 provided with an inlet valve 51 and a sleeve 4 which serves to sealingly guide the actuating pin T are securely mounted. The barrel part 5 is held between the bottom part 6 and the sleeve 4. The bottom part 6 can be permanently snap fitted into openings formed by the bushing 36 of the pump body 3. The design of these fixed parts 4, 5 and 6 is not critical for the present invention, and so other architectures may be envisaged without departing from the scope of the invention.

The actuating pin T is slidably mounted inside the pump body 3 along a longitudinal axis X. A portion of the actuating pin T emerges upwards from the pump body 3: this emergent portion receives an actuator P, on which the user can push in order to displace the actuating pin T axially back and forth inside the pump body 3. In FIG. 1, the actuator P is a nasal-type actuator, but any actuator may be mounted on the actuating pin T. The actuator P is not critical to the present invention.

The actuating pin T here is of a particular type, since it comprises a valve shutter 1 in the upper portion and a piston member 2 in the lower portion. These two parts are connected to one another with axial freedom of movement along the axis X. However, they are fixedly connected to one another in rotation, so that rotation of the valve shutter 1 causes rotation of the piston member 2. And since the actuator P is securely mounted on the valve shutter 1, the rotation of the actuator P causes the actuating pin T as a whole to rotate.

More specifically, the valve shutter 1 comprises a tube 11 which extends upwards from a base 13. The valve shutter 1 can be better seen in FIGS. 4a 4b and 4c. The tube 11 forms a free end on which the actuator P is securely mounted both axially and in rotation. It should be noted that the tube 11 forms a venting groove 12, the largest dimension of which is axial. This venting groove 12 is located in the proximity of the base 13. Its function will be explained below. The base 13 has an increased diameter compared with that of the tube 11. The base 13 firstly comprises two longitudinal axial ribs 14 which project radially outwards. The two ribs 14 are disposed diametrically opposite one another. It should also be noted that the venting groove 12 is located axially just above one of the ribs 14. Below the ribs 14, the base 13 forms an annular groove 15, which serves to receive an O-ring G15, which can be seen in FIG. 1. Internally, the base 13 forms a housing 16 for another O-ring G16, also visible in FIG. 1. The valve shutter 1 is traversed internally by a through duct 10 which extends from the housing 16 to the free upper end of the tube 11 on which the actuator P is mounted.

The piston member 2 comprises a head 21 and a tube 25. The piston member 2 can be seen better in FIGS. 5a, 5b, 5c and 5d. The head 21 forms a helical furrow 22 the function of which will be provided below. Below this helical furrow 22, the head 21 forms a groove 23 for receiving an O-ring G23, which can be seen in FIG. 1. Internally, the head 21 defines a receiving housing 20, which forms two longitudinal axial recesses 24, intended to receive the two ribs 14 of the valve shutter 1, in a manner such as to lock the two parts against rotation with respect to one another. The recesses 24 have dimensions which are very slightly greater than those of the ribs 14 in a manner such as to be able to receive them without play or excessive friction, so that the two parts can still be displaced axially by sliding. At its bottom end, the tube 25 forms an inner housing 27 for an O-ring G27. At its top end, the tube 25 internally forms a movable valve member 26, which will cooperate with the O-ring G16 of the valve shutter 1, in a manner such that they together form an outlet valve for the pump. As can be seen in FIG. 5d, the receiving housing 20 communicates with the inside of the tube 25 via passages formed around the movable valve member 26, which is connected to the tube 25 by bridges of material.

The pump body 3 can be seen better in FIGS. 6a, 6b and 6c. The pump body 3 internally defines a sliding barrel 31, which has the particular feature of forming a helical thread 32 at its upper end. Above this helical thread 32, the pump body forms an axial abutment 33, internally defining a housing 34 for an O-ring G34, which can be seen in FIG. 1. Below the sliding barrel 31, the body 3 forms the fixing ring 35, already described, as well as the assembly bushing 36.

Reference will now be made again to FIG. 1, as well as to FIGS. 2a and 3a in order to explain the cooperation of these various parts within the pump of the invention. As already mentioned, the base 13 of the valve shutter 1 is axially engaged inside the receiving housing 20 of the piston member 2, with the ribs 14 engaged by sliding inside recesses 24 of the receiving housing 20. The maximum engagement of the base 13 inside the receiving housing 20 brings about a sealed contact between the O-ring G23 and the wall of the receiving housing 20, as well as a sealed contact between the O-ring G16 and the movable valve member 26. A slight axial displacement of these two parts from this position of maximum engagement leads to the separation of the seal G16 from the movable valve member 26, while the O-ring G23 remains in sealed contact with the internal wall of the receiving housing 20. In other words, the neck seal G16 is detached, while the O-ring G23 slides in a sealed manner. Thus, a seal between these two parts is always maintained. This actuating pin T, constituted by the assembly of the valve shutter 1 with the piston member 2, is integrated into the pump body 3 in the following manner. In the locked inoperative state of the pump, the head 21 of the piston member 2 is in threaded engagement with the barrel 31. More precisely, the helical thread 32 of the body 3 is in engagement with the helical furrow 22 of the head 21 of the piston member 2. In this locked inoperative state, it should be noted that the head 21 is in contact with the abutment 33. However, the O-ring G23 still remains in sealed sliding contact inside the barrel 31. The tube 25 of the piston member 2 is engaged inside the sleeve 4 and slides in a sealed manner because of the O-ring G4. In addition, the tube 25 is also engaged around the barrel 52 of the barrel part 5 with a sliding, sealed contact provided by the O-ring G27. Thus, a fluid product chamber Cp is created between the inlet valve 51 and the outlet valve formed by the O-ring G16 and the movable valve member 26. It can be said that the piston of the fluid product chamber Cp is formed by the O-ring G27 supported by the pipe 25.

The valve shutter 1, the base 13 of which is engaged in the head 21 of the piston member 2, protrudes with its tube 11 through the abutment 33. It should be noted that the base 13 may also come into contact with the abutment 33. The O-ring G34 surrounds the tube 11: however, in this locked inoperative position, it should be noted in FIG. 3a that the sealed contact between the O-ring G34 and the tube 11 is broken by the venting groove 12, which is located axially at the O-ring G34.

From this locked inoperative position, shown in FIGS. 1, 2a and 3a, the user can grasp the actuator P and drive it in rotation, thereby causing the piston member 2 to turn inside the pump body 1 via the valve shutter 1. In fact, the torque applied to the actuator P is transmitted by the valve shutter 1 to the piston member 2, because of the lock against rotation obtained by the engagement of the ribs 14 in the recesses 24. This rotation applied to the actuator P causes actuation of the threaded connection created between the helical furrow 22 and the helical thread 32. As a result, the actuator P and the actuating pin T are displaced axially downwards. This is shown in FIGS. 2b and 3b. The helical thread 32 is completely disengaged from the helical furrow 22 and the head 21 with its O-ring G23 can then slide axially in a sealed manner in the barrel 31 of the pump body 3 by pushing axially on the actuator P. The space thus formed between the head 21 and the abutment 33 constitutes a partial vacuum chamber Cd, which emerges with the rotation of the actuator P and which is increased further by pushing axially on the actuator P. Because of the O-rings G23 and G34, the partial vacuum chamber Cd is isolated from the outside in a manner such that an increasing partial vacuum is produced as the head 21 moves away from the abutment 33. Simultaneously, the O-ring G27 slides along the barrel 52, thereby reducing the volume of the pump chamber Cp and increasing the pressure on the fluid product. In response to this increase in pressure, a slight axial displacement is created between the piston member 2 and the valve shutter 1, in a manner such as to separate the O-ring G16 from the movable valve member 26, thereby creating an outlet passage for the fluid product through the valve shutter 1 and the actuator P. The operation of this differential piston will not be described in greater detail here, given that it is known from the prior art.

Once the actuator P is fully depressed, dispensing of the fluid product is complete and the user then releases the pressure exerted on the actuator P. The partial vacuum created inside the partial vacuum chamber Cd has the effect of returning the valve shutter pin T to the inoperative position. In other words, the partial vacuum created in the partial vacuum chamber Cd urges the head 21 of the piston member 2 in the direction of the abutment 33. The O-ring G23 is therefore displaced in a sliding and sealed manner inside the barrel 31 until the head 21 comes into abutment against the beginning of the helical thread 32. The user then knows that it is necessary to lock the pump again by rotating the actuator, which has the effect of interlocking the helical thread 32 with the helical furrow 22. The end of the rotation, corresponding to the locked inoperative position, is reached when the head 21 again comes into contact against the abutment 33. The configuration has then returned to that shown in FIGS. 1, 2a and 3a, in which the partial vacuum chamber Cd, reduced to a state of minimum volume, can communicate with the outside because of the lack of sealing generated by the venting groove 12.

Thus, the travel of the actuating pin P makes it possible to vary the volume of the partial vacuum chamber Cd between a state of minimum volume, or even zero volume, and a state of maximum volume. The travel of the head 21, which can be likened to a partial vacuum chamber piston, with its O-ring G23 acting as a piston lip, can be broken down into two parts, namely a major travel corresponding to sealed sliding without a rotary component and a minor travel corresponding to the threaded engagement of the thread 32 in the furrow 22. The major travel is generated by the partial vacuum prevailing in the partial vacuum chamber Cd, whereas the minor travel is generated by manually driving the actuator P in rotation. In other words, the major travel is solely pneumatic, whereas the minor travel is essentially mechanical. It is thus possible to qualify the threaded engagement between the thread 32 and the furrow 22 as mechanical means, as opposed to the pneumatic means of the major travel. The mechanical means, formed by the thread 32 and the furrow 22, make it possible to return the actuating pin T in a reliable manner to the locked inoperative position, in which the partial vacuum chamber Cd, reduced to its state of minimum volume, communicates with the outside via the venting groove 12. It will readily be understood, by reference to FIG. 3a, that this communication with the outside is very quickly broken as soon as the venting groove 12 is no longer located axially at the O-ring G34. A travel of a few tenths of a millimetre is enough to break this communication and to isolate the partial vacuum chamber Cd from the outside. The venting of the partial vacuum chamber Cd makes it possible to reset the pressure each time the pump is actuated. Thus, even in the case in which the partial vacuum chamber Cd might be leaking, and thus might allow air to enter the interior, this air would be evacuated at the end of actuation because of the venting of the chamber Cd through the venting groove 12.

In the absence of mechanical means, constituted by the thread 32 and the furrow 22, the operation of the pump is possible, but in this case it would be necessary for the partial vacuum inside the partial vacuum chamber Cd to be sufficient to bring the head 21 into contact with the abutment 33, in a manner such as to re-establish communication with the outside through the groove 12. Conversely, the pump could operate without a venting groove 12, but in this case there would have to be no leakage at the partial vacuum chamber Cd. Clearly, the cumulative employment of mechanical means and venting is particularly advantageous and constitutes the preferred embodiment.

The pump described here incorporates a piston member 2 which can be described as a differential piston since it is displaced not only by pushing on the actuator, but also in response to a threshold pressure inside the fluid product chamber Cp. Without departing from the scope of the invention, it is possible to employ the partial vacuum chamber Cd of the invention in a more conventional pump, in which the piston of the partial vacuum chamber is securely mounted on the actuating pin, while the piston of the fluid product chamber Cp is slidably mounted on the actuating pin.

Without departing from the scope of the invention, it is possible to provide a plurality of venting grooves 12 and one, or more than two, ribs 14. The O-rings employed on different parts could be replaced by monobloc lips.

Because of the invention, it is possible to replace the metal or plastic spring of the prior art by an air spring, part of the travel of which results from the actuation of the actuator, which is preferably rotary. However, it is also possible to envisage displacing the actuator in the axial direction alone. Regarding venting the partial vacuum chamber Cd, it allows a reset on each actuation.

1. A pump comprising a fluid product chamber with a variable volume, as well as an actuating pin which is axially displaceable between an inoperative position and a depressed position, in a manner such as to vary the volume of the fluid product chamber, respectively between a state of maximum volume and a state of minimum volume, the pump comprising a return means for returning the actuating pin to the inoperative position, corresponding to the state of maximum volume for the fluid product chamber the actuating pin being provided with an actuator which can be actuated by a user,
in which the return means comprises a partial vacuum chamber with a volume which is variable between a state of minimum volume and a state of maximum volume by displacement of the actuating pin the state of minimum volume of the partial vacuum chamber being reached in the inoperative position of the actuating pin and the state of maximum volume of the partial vacuum chamber being reached in the depressed position of the actuating pin the state of maximum volume of the partial vacuum chamber corresponding to a maximum partial vacuum and the state of minimum volume of the partial vacuum chamber corresponding to a minimum partial vacuum, the actuating pin being urged towards its inoperative position by the partial vacuum prevailing in the partial vacuum chamber,
in which mechanical means which are independent of the partial vacuum in the partial vacuum chamber, can be actuated by the user in order to return and maintain the partial vacuum chamber in its state of minimum volume,
wherein the mechanical means comprise a threaded connection, allowing displacement of the actuating pin and the actuator both axially and in rotation.

2. The pump as claimed in claim 1, in which the mechanical means are controlled by the actuator via the actuating pin.

3. The pump as claimed in claim 1, or in which the threaded connection defines an axial travel which is delimited on one side by a top abutment corresponding to the minimum volume state of the partial vacuum chamber and on the other side by a release of the threaded connection allowing subsequent variation of the volume of the partial vacuum chamber towards its maximum volume state.

4. The pump as claimed in claim 1, in which the partial vacuum chamber communicates with the outside in its minimum volume state.

5. The pump as claimed in claim 1, in which the actuating pin comprises a partial vacuum chamber piston which slides axially in a sealed manner in a barrel of the partial vacuum chamber in a manner such as to define the partial vacuum chamber between them, the actuating pin forming a venting groove which brings the partial vacuum chamber into communication with the outside in the inoperative state of the actuating pin.

6. The pump as claimed in claim 1, in which the actuating pin comprises a valve shutter and a piston member the piston member forming the partial vacuum chamber piston sliding in the barrel of the partial vacuum chamber, the piston member also forming a fluid product chamber piston sliding in a fluid product chamber barrel, the piston member advantageously forming a movable outlet valve member for the fluid product chamber and the valve shutter advantageously forming an outlet valve seat for the fluid product chamber.

7. The pump as claimed in claim 6, in which the piston member is axially movable with respect to the valve shutter under the effect of the pressure prevailing in the fluid product chamber but is driven in rotation by the valve shutter.

8. The pump as claimed in claim in which the threaded connection is located between the piston member and the barrel of the partial vacuum chamber.

9. The pump as claimed in claim 1, in which the partial vacuum chamber defines a maximum axial travel between its minimum volume state and its maximum volume state, this maximum axial travel being broken down into:
a major travel starting from the maximum volume state, which is induced by the partial vacuum prevailing in the partial vacuum chamber and
a minor travel to the state of maximum volume, which is induced by driving the actuator in rotation and/or axially, which ends in a position of axial locking of the actuator.
